(19) United States
(12) Patent Application Publication
(10) Pub. No.: US 2026/0240746 A1
(43) Pub. Date: Aug. 20, 2026

(54) TOPICAL COMPOSITION AND IMPLEMENTATIONS THEREOF

(71) Applicant: ULTRAMARINE & PIGMENTS LIMITED, MUMBAI (IN)

(72) Inventors: Renuka THERGAONKAR, Thane (IN); Deepak ROHRA, Mumbai (IN); Shraddha SURESH, Mumbai (IN); Gnanaprakash AYYAPPAN, Arni (IN); Pavankumar ADURI, Kalyan Maharashtra (IN); Tara PARTHASARATHY, Chennai (IN)

(21) Appl. No.: 18/869,790

(22) PCT Filed: Mar. 21, 2023
(86) PCT No.: PCT/IN2023/050275

§ 371 Date: Nov. 26, 2024

(30) Foreign Application Priority Data
May 27, 2022 (IN) 202221030642

(51) Int. Cl. A61K 8/67; A61K 8/37; A61Q 5/00; A61Q 19/00

(52) U.S. Cl. CPC A61K 8/678; A61K 8/37; A61Q 5/00; A61Q 19/007; A61K 2800/51; A61K 2800/524

(57) ABSTRACT

The present disclosure provides a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids. The present disclosure provides a process for preparing the topical composition. The present disclosure also provides a skin-care and a hair-care topical composition.

### FIELD OF INVENTION

The present disclosure broadly relates to the field of cosmetics. Particularly, the present disclosure relates to a topical composition for skin care and hair care. The present disclosure also relates to a process for preparing the topical composition.

### BACKGROUND OF INVENTION

The cosmetic industry, globally, is moving towards safe, natural, sustainable, and conscious cosmetics. Synthetic ingredients are not preferred in the cosmetic industry, especially in skin and hair care. The beauty industry is continuously looking at alternatives for synthetic ingredients, such as parabens, phthalates, sodium lauryl sulphate, and silicones, as they are concerned about the perceived health risks.

Silicones are a family of polymers ranging from low viscosity fluids to viscous gums, to cross-linked elastomers and hard resins. Silicones are derived from silica through an energy intensive process and further undergo extensive chemical processing before addition into cosmetic products. Dimethicone, cyclomethicone, cyclohexasiloxane, cetearylmethicone, cyclopentasiloxane are some of the commonly used silicone ingredients in cosmetics. In spite of the versatile nature of silicones in providing a conditioning effect on skin and hair, silicones are not preferred by consumers as they are occlusive ingredients. The protective layer they form on skin offers a momentary pleasant sensorial experience, however, the occlusive nature may worsen congestive skin types by trapping dirt and clogging pores. In the long run, silicones may not contribute to overall health and improvement of the skin. There are other disadvantages to using silicones in hair care products. The most common one is silicone prevents the hair follicles from obtaining oxygen thereby resulting in hair loss. Certain silicone derivatives like cyclomethicones cause environmental damage and are strictly restricted for use in personal care products.

There is a dire need in the state of art for topical compositions that are silicone free, non-toxic and biocompatible, that moisturize and provide nourishment to the skin and hair, and are easily biodegradable.

### SUMMARY OF THE INVENTION

In an aspect of the present disclosure, there is provided a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids.

In another aspect of the present disclosure, there is provided a process for preparing a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids, said process comprising contacting stoichiometric ratio of a triglyceride, an ester of C₈ to C₁₆ fatty acid, and vitamin E under stirring at a speed in a range of 50 to 1200 rpm at a temperature in a range of 20 to 60° C., wherein the triglyceride is a medium chain triglyceride comprising C₆ to C₁₂ fatty acids.

In further aspect of the present disclosure, there is provided a cosmetic formulation comprising a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids with an additive.

In another aspect of the present disclosure, there is provided a method of treating keratinous tissue of a subject, the method comprising topically applying the composition or the cosmetic formulation as described herein to the keratinous tissue.

In one more aspect of the present disclosure, there is provided a method of treating hair, the method comprising topically applying to hair in need thereof, the composition or the cosmetic formulation as described herein, wherein topical application of the composition or formulation to the hair treats the hair.

In another aspect of the present disclosure, there is provided a method of moisturizing skin, comprising applying an effective amount of the composition or the cosmetic formulation as described herein to the skin.

In other aspect of the present disclosure, there is provided a cosmetic kit comprising a) a first compartment comprising the composition or the cosmetic formulation as described herein; and b) a second compartment comprising an accessory tool.

In another aspect of the present disclosure, there is provided a skin-care topical composition comprising: (a) 70 to 95% (w/w) of a triglyceride; (b) 1 to 15% (w/w) of isopropyl myristate; and (c) 0.1 to 0.3% (w/w) of vitamin E, wherein the triglyceride is a medium chain triglyceride comprising C₆ to C₁₂ fatty acids.

In one more aspect of the present disclosure, there is provided a hair-care topical composition comprising: (a) 70 to 95% (w/w) of a triglyceride; (b) 1 to 15% (w/w) of caprylate caprate; and (c) 0.1 to 0.3% (w/w) of vitamin E, wherein the triglyceride is a medium chain triglyceride comprising C₆ to C₁₂ fatty acids.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 illustrates A) the cumulative scores for sample 1(A_89.8) by volunteers and B) the overall cumulative score of the sensory parameters for sample 1(A_89.8) against silicones (cyclopentasiloxane, dimethicone), in accordance with an implementation of the present disclosure.

FIG. 2 illustrates the moisturization analysis of A) sample 1(A_89.8) in comparison to silicones B) cyclopentasiloxane, and C) dimethicone, in accordance with an implementation of the present disclosure.

FIG. 3 illustrates the zone of inhibition formation in agar diffusion test of murine fibroblast cell line (3T3) cells in response to A) sample 1(A_89.8) along with B) negative control and C) positive control samples, in accordance with an implementation of the present disclosure.

FIG. 4 illustrates the dose dependent viability induced by A) reference standard and B) sample 1(A_89.8) in murine fibroblast cell line (3T3) cells, in accordance with an implementation of the present disclosure.

FIG. 5 illustrates A) the cumulative scores for sample 2 (B_89.8) and B) the overall cumulative score of all the sensory parameters for sample 2 (B_89.8) against silicones (cyclopentasiloxane, dimethicone), in accordance with an implementation of the present disclosure.

FIG. 6 illustrates the moisturization analysis of A) sample 2(B_89.8) in comparison to silicones B) cyclopentasiloxane, and C) dimethicone, in accordance with an implementation of the present disclosure.

FIG. 7 illustrates the zone of inhibition formation in agar diffusion test of murine fibroblast cell line (3T3) cells in response to A) sample 2(B_89.8) along with B) negative control and C) positive control samples, in accordance with an implementation of the present disclosure.

FIG. 8 illustrates the dose dependent viability induced by A) sample 2 (B_89.8) and B) reference standard in murine fibroblast cell line (3T3) cells, in accordance with an implementation of the present disclosure.

FIG. 9 illustrates the overall cumulative score of the sensory parameters for sample 3(A_74.8) against commercial silicones (cyclopentasiloxane, dimethicone), in accordance with an implementation of the present disclosure.

FIG. 10 illustrates the overall cumulative score of the sensory parameters for sample 4(B_74.8) against silicones (cyclopentasiloxane, dimethicone), in accordance with an implementation of the present disclosure.

FIG. 11 illustrates the moisturization analysis of sample 3(A_74.8), sample 4(B_74.8), in accordance with an implementation of the present disclosure.

FIG. 12 illustrates the overall cumulative score of the sensory parameters for sample 5(A_59.8) against silicones (cyclopentasiloxane, dimethicone), in accordance with an implementation of the present disclosure.

FIG. 13 illustrates the overall cumulative score of the sensory parameters for sample 6(B_59.8) against silicones (cyclopentasiloxane, dimethicone), in accordance with an implementation of the present disclosure.

FIG. 14 illustrates the moisturization analysis of sample 5(A_59.8), sample 6(B_59.8), in accordance with an implementation of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “topical” used herein, relates to the use of a composition or a formulation as described herein, either alone or incorporated in a suitable form, and applied at the site of keratinous tissue, hair, or skin exteriorly for exertion of local action.

The term “sensorial analysis” refers to a method to analyse and interpret the characteristics of a formulation as perceived by the five senses such as seeing, hearing, touching, tasting, and smelling.

The term “moisturization analysis” refers to the method of measuring the moisture content before and after application of the composition.

The term “fatty acid” refers to carboxylic acids with a long saturated or unsaturated aliphatic chain. The number of carbon atoms of the fatty acids could vary from 4 to 28. In particular, in the present disclosure, the fatty acids have carbon atoms in the range of C₆ to C₁₆ which may be saturated, unsaturated, or partially saturated and may be straight chain or branched. The term “fatty acid” of the present disclosure includes but not limited to myristic acid, capric acid, caprylic acid, caproic acid, lauric acid, or combinations thereof. The term “ester of fatty acid” refers to esters of C₈ to C₁₆ fatty acid especially esters of myristic acid, caprylic/capric acid. The term “ester of fatty acid” includes isopropyl myristate and caprylate caprate. The terms caprylate caprate and coco caprylate caprate are used interchangeably and refers to an ester of natural fatty alcohol of caprylic and capric acid. The term “medium chain fatty acids” refers to fatty acids having 6 to 12 carbon atoms.

The term “medium chain triglycerides” (MCT) refers to triglycerides of fatty acids of medium carbon chain. In other words, medium chain triglycerides are glycerol esters of two or three fatty acids having 6 to 12 carbon chain which are aliphatic and saturated. MCT of the present disclosure has an acid value of less than 0.1 and colour index of less than 50.

The term “acid value” refers to number of milligrams of a base (potassium hydroxide) required to neutralize one gram of fatty acid in a sample. In the present disclosure, acid value refers to number of milligrams of base necessary to neutralize one gram of free fatty acid present in MCT. The lower acid value indicates MCT is almost completely esterified and there is a minimum of free fatty acids that are present. This further indicated compatibility of MCT for use in a topical composition or a cosmetic formulation.

The term “colour index” refers to the colour scale or the yellowness index to assess the quality of liquids that are clear to yellowish in colour. The scale ranges from distilled water at 0 to a yellow solution of 500 (parts per million of platinum cobalt to water). The colour index of zero is clear white or water white and 500 is light yellow. In the present disclosure, colour index of MCT is less than 50 which means MCT is fairly colourless.

The term “Vitamin E” is a fat-soluble vitamin of several forms, which includes four tocopherols and four tocotrienols. Vitamin E is a powerful antioxidant that neutralizes harmful free radicals and protects skin from ultraviolet radiation damage.

The term “keratinous tissue” used herein refers to tissue having keratin which is a structural fibrous protein also known as scleroprotein. In the present disclosure, the term “keratinous tissue” refers to hair and skin.

The term “RH” refers to relative humidity which is a percentage that indicates the amount of water vapor in the air at a given temperature compared to the maximum amount of water vapor amount at that same temperature. In other words, relative humidity also refers to the ratio of the partial pressure of water vapor to the equilibrium vapor pressure of water at the same temperature. In the present disclosure, the chemical stability of the composition is evaluated at 45° C. under 70% RH conditions.

The term “Murine fibroblast cell line (3T3)” refers to mouse origin fibroblast cells, which are the most common cells of connective tissue in animals. In the present disclosure, murine fibroblast cell line (3T3) is used as a skin model to study cytocompatibility and skin reactivity of the composition, as skin is primarily made of connective tissues.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature range of 20° C. to 60° C. should be interpreted to include not only the explicitly recited limits of −20° C. to 60° C., but also to include sub-ranges, such as 25° C. to 35° C., 45° C. to 55° C., and so forth, as well as individual amounts, including fractional amounts, within the specified ranges, such as 40° C., 50° C., 53.5° C., and 55° C., for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described.

The present disclosure is not to be limited in scope by the specific embodiments described herein, which are intended for the purposes of exemplification only. Functionally equivalent products, compositions, and methods are clearly within the scope of the disclosure, as described herein.

As discussed above, there are various limitations associated with the conventional cosmetic and personal care products. The available cosmetic products comprise synthetic products, particularly silicone, which forms a protective but occlusive film that prevents the skin to perspire and breathe naturally, thereby clogging pores and further, worsening congestive skin types. Further, silicones as part of hair care products prevents follicle from obtaining oxygen thereby causing hair loss. Moreover, silicone containing products remains questionable in terms of safety and toxicity. Most of the silicone containing cosmetic products are found to be non-biodegradable and harmful to the environment at large. Currently, the world is on a green movement with people preferring to use plant originated, natural ingredients in all products associated with health and human welfare. Hence, cosmetic, and personal care products that are silicone free are favored over conventional products.

Therefore, the present disclosure solves the problems existing in the art by providing a composition that is based on the combination of the naturally derived ingredients that results in the formation of a film on the skin and helps in enhancing the moisturizing and nourishing ability and overall, it enhances the sensorial feel. The composition of the present disclosure is a silicone-free composition that circumvents the problems existing in the conventional cosmetic composition containing silicones.

Accordingly, the present invention provides a topical composition comprising triglycerides, ester of C₈ to C₁₆ fatty acid and vitamin E. Medium chain triglycerides (MCT) are obtained as an esterification product of glycerol with medium-chain fatty acids. These MCTs are suitable to be added as a component in a cosmetic composition which is capable of providing soothing and non-greasy emollient with neutral sensory characteristics. MCTs also provides a long shelf life and hence is also useful as a carrier oil and it does not interfere with the scent of aromatic blends, absorbs rapidly, does not stain clothing, and leaves behind a silky but non-greasy feeling to the skin. The esters of C₈ to C₁₆ fatty acid is isopropyl myristate or caprylate caprate. Isopropyl myristate which is an ester of isopropyl alcohol with myristic acid is a polar emollient and used in the composition wherein skin absorption is desired and is a skin enhancer. Caprylate caprate or coco caprylate caprate is an ester of natural fatty alcohol, caprylic acid and capric acid and is 100% derived from natural, renewable feedstock. It has unique chemical composition and chain length distribution, making it lighter than traditional grades of coco caprylate/caprate esters. Caprylate caprate provides enhanced sensorial effect and is highly stable. Caprylate caprate is also capable for fast spreading and provides low greasiness. Thus due to said advantages and being derived from natural resources MCT, isopropyl myristate/caprylate caprate are suitable alternatives for silicones in cosmetics. Vitamin E is an anti-oxidant and additionally provides better absorption on skin thereby providing nourishment to the skin and hair. The other anti-oxidants such as butylated hydroxy anisole, butylated hydroxytoluene do not exhibit such absorption properties and thus is not a preferred component in a topical composition. Accordingly, the topical composition of the present disclosure comprises medium chain triglycerides, isopropyl myristate or coco caprylate caprate and vitamin E and the composition is an effective and synergistic composition for enhanced skin retention, nourishing emollient layer, increasing moisture level, and enhancing sensorial feel. The components of the composition have moisturizing and nourishing abilities and overall is safe, non-comedogenic, non-toxic, silicone free and biodegradable which provides hydrated or moisturizing effect.

In an embodiment of the present disclosure, there is provided a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids.

In an embodiment of the present disclosure, there is provided a topical composition comprising (a) 75 to 92% (w/w) of a triglyceride; (b) 5 to 12% (w/w) of an ester of C₈ to C₁₆ fatty acid; and (c) 0.15 to 0.25% (w/w) of vitamin E, wherein the triglyceride is a medium chain triglyceride comprising C₆ to C₁₂ fatty acids. In another embodiment of the present disclosure, there is provided a topical composition comprising (a) 89 to 90% (w/w) of a triglyceride; (b) 9 to 11% (w/w) of an ester of C₈ to C₁₆ fatty acid; and (c) 0.18 to 0.22% (w/w) of vitamin E, wherein the triglyceride is a medium chain triglyceride comprising C₆ to C₁₂ fatty acids. In yet another embodiment of the present disclosure, there is provided a topical composition comprising (a) 89.8% (w/w) of a triglyceride; (b) 10% (w/w) of an ester of C₈ to C₁₆ fatty acid; and (c) 0.2% (w/w) of vitamin E, wherein the triglyceride is a medium chain triglyceride comprising C₆ to C₁₂ fatty acids.

In an embodiment of the present disclosure, there is provided a topical composition as described herein, wherein the ester of C₈ to C₁₆ fatty acid is isopropyl myristate or caprylate caprate.

In an embodiment of the present disclosure, there is provided a topical composition as described herein, wherein the triglyceride has an acid value of less than 0.1 and colour index of less than 50.

In an embodiment of the present disclosure, there is provided a topical composition comprising (a) 70 to 95% (w/w) of a medium chain triglyceride comprising C₆ to C₁₂ fatty acids; (b) 1 to 15% (w/w) of isopropyl myristate or caprylate caprate; and (c) 0.1 to 0.3% (w/w) of vitamin E, wherein the triglyceride is a medium chain triglyceride comprising C₆ to C₁₂ fatty acids having an acid value of less than 0.1 and colour index of less than 50.

In an embodiment of the present disclosure, there is provided a topical composition as described herein, wherein the composition is for skin-care or hair-care.

In an embodiment of the present disclosure, there is provided a topical composition as described herein, wherein the composition is chemically stable for at least 3 months at 45° C., as measured at 70% RH.

In an embodiment of the present disclosure, there is provided a topical composition as described herein, wherein the composition is stable and exhibits cytocompatibility.

In an embodiment of the present disclosure, there is provided a topical composition for skin-care or hair-care the composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids; and the composition is chemically stable for at least 3 months at 45° C., as measured at 70% RH and exhibits cytocompatibility.

In an embodiment of the present disclosure, there is provided a topical composition as described herein, wherein the composition further comprises a component selected from antimicrobial, antibacterial, antifungal, antiprotozoal, and antiviral agents.

In an embodiment of the present disclosure, there is provided a topical composition for skin-care or hair-care the composition comprising (a) 70 to 95% (w/w) of a triglyceride; (b) 1 to 15% (w/w) of an ester of C₈ to C₁₆ fatty acid; (c) 0.1 to 0.3% (w/w) of vitamin E and (d) a component selected from antimicrobial, antibacterial, antifungal, antiprotozoal, and antiviral agents wherein the triglyceride is a medium chain triglyceride comprising C₆ to C₁₂ fatty acids.

In an embodiment of the present disclosure, there is provided a process for preparing a topical composition comprising (a) 70 to 95% (w/w) of a triglyceride; (b) 1 to 15% (w/w) of an ester of C₈ to C₁₆ fatty acid; and (c) 0.1 to 0.3% (w/w) of vitamin E, wherein the triglyceride is a medium chain triglyceride comprising C₆ to C₁₂ fatty acids, said process comprising contacting stoichiometric ratio of a triglyceride, an ester of C₈ to C₁₆ fatty acid, and vitamin E under stirring at a speed in a range of 50 to 1200 rpm at a temperature in a range of 20 to 60° C. In another embodiment of the present disclosure, wherein contacting stoichiometric ratio of a triglyceride, an ester of C₈ to C₁₆ fatty acid, and vitamin E is carried out under stirring at a speed in a range of 100 to 1000 rpm at a temperature in a range of 25 to 50° C. In yet another embodiment of the present disclosure, wherein contacting stoichiometric ratio of a triglyceride, an ester of C₈ to C₁₆ fatty acid, and vitamin E is carried out under stirring at a speed of 150 rpm at a temperature of 27° C. In one another embodiment of the present disclosure, wherein contacting stoichiometric ratio of a triglyceride, an ester of C₈ to C₁₆ fatty acid, and vitamin E is carried out for a time period in a range of 10 to 60 minutes.

In an embodiment of the present disclosure, there is provided a cosmetic formulation comprising (a) 70 to 95% (w/w) of a triglyceride; (b) 1 to 15% (w/w) of an ester of C₈ to C₁₆ fatty acid; (c) 0.1 to 0.3% (w/w) of vitamin E; and (d) an additive, wherein the triglyceride is a medium chain triglyceride comprising C₆ to C₁₂ fatty acids.

In an embodiment of the present disclosure, there is provided a cosmetic formulation as described herein, wherein the additive is selected from a preservative, a pH adjusting agent, a chelating agent, or a co-solvent. In another embodiment of the present disclosure, the cosmetic formulation is topically applied on hair or skin.

In an embodiment of the present disclosure, there is provided a cosmetic formulation comprising (a) 70 to 95% (w/w) of a triglyceride; (b) 1 to 15% (w/w) of an ester of C₈ to C₁₆ fatty acid; (c) 0.1 to 0.3% (w/w) of vitamin E; and (d) an additive selected from a preservative, a pH adjusting agent, a chelating agent, or a co-solvent, wherein the triglyceride is a medium chain triglyceride comprising C₆ to C₁₂ fatty acids.

In an embodiment of the present disclosure, there is provided a method of treating keratinous tissue of a subject, the method comprising topically applying the composition or the cosmetic formulation as described herein to the keratinous tissue. In another embodiment of the present disclosure, wherein the keratinous tissue is skin or hair.

In an embodiment of the present disclosure, there is provided a method of treating hair, the method comprising topically applying to hair in need thereof, the composition or the cosmetic formulation as described herein, wherein topical application of the composition or formulation to the hair treats the hair.

In an embodiment of the present disclosure, there is provided a method of moisturizing skin, the said method comprising applying an effective amount of the composition or the cosmetic formulation as described herein to the skin.

In an embodiment of the present disclosure, there is provided a cosmetic kit, said cosmetic kit comprising a) first compartment comprising the topical composition or the cosmetic formulation as described herein; and b) a second compartment comprising an accessory tool. In one embodiment of the present disclosure, the cosmetic kit further comprises a user manual. In another embodiment of the present disclosure, the cosmetic kit comprises of a third compartment comprising cleansing agents including but not limited to sulfates, sulfonates, glucosides, taurates, betaines, cocamidopropyl betaine, amides, and so on.

In an embodiment of the present disclosure, there is provided a skin-care topical composition comprising: (a) 70 to 95% (w/w) of a triglyceride; (b) 1 to 15% (w/w) of isopropyl myristate; and (c). 0.1 to 0.3% (w/w) of vitamin E, wherein the triglyceride is a medium chain triglyceride comprising C₆ to C₁₂ fatty acids. In another embodiment of the present disclosure, there is provided a skin-care topical composition comprising: (a) 75 to 90% (w/w) of a triglyceride; (b) 5 to 12% (w/w) of isopropyl myristate; and (c). 0.15 to 0.3% (w/w) of vitamin E, wherein the triglyceride is a medium chain triglyceride comprising C₆ to C₁₂ fatty acids. In one another embodiment of the present disclosure, there is provided a skin-care topical composition comprising: (a) 89.8% (w/w) of a triglyceride; (b) 10% (w/w) of isopropyl myristate; and (c) 0.2% (w/w) of vitamin E, wherein the triglyceride is a medium chain triglyceride comprising C₆ to C₁₂ fatty acids.

In an embodiment of the present disclosure, there is provided a hair-care topical composition comprising: (a) 70 to 95% (w/w) of a triglyceride; (b). 1 to 15% (w/w) of caprylate caprate; and (c) 0.1 to 0.3% (w/w) of vitamin E, wherein the triglyceride is a medium chain triglyceride comprising C₆ to C₁₂ fatty acids. In another embodiment of the present disclosure, there is provided a hair-care topical composition comprising: (a) 75 to 90% (w/w) of a triglyceride; (b) 5 to 12% (w/w) of caprylate caprate; and (c) 0.15 to 0.3% (w/w) of vitamin E, wherein the triglyceride is a medium chain triglyceride comprising C₆ to C₁₂ fatty acids. In yet another embodiment of the present disclosure, there is provided a hair-care topical composition comprising: (a) 89.8% (w/w) of a triglyceride; (b) 10% (w/w) of caprylate caprate; and (c) 0.2% (w/w) of vitamin E, wherein the triglyceride is a medium chain triglyceride comprising C₆ to C₁₂ fatty acids.

In an embodiment of the present disclosure, there is provided a topical composition, or a cosmetic formulation as described herein, wherein the composition or the formulation is effective in treating skin and hair and providing moisturizing effect.

In an embodiment of the present disclosure, there is provided a topical composition, or a cosmetic formulation as described herein, wherein the composition is in the form of cream, gel, lotion, or ointment.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

### Materials and Methods

Medium chain fatty acids, glycerol, isopropyl myristate, caprylate caprate (coco caprylate caprate) and vitamin E used were all procured commercially. Medium chain triglycerides is obtained as an esterification product of glycerol and medium chain fatty acids. MCT used in the present disclosure has acid value of less than 0.1 and colour index of less than 50. Positive control refers to sodium dodecyl sulphate and negative control refers to phosphate buffered saline.

For comparative purposes commercially available silicone compositions dimethicone, and cyclopentasiloxane were used.

### Example 1

### Preparation of Topical Compositions

Different topical compositions were prepared by contacting stoichiometric ratio (Table 1) of a medium chain triglyceride, isopropyl myristate or coco caprylate caprate, and vitamin E under stirring at a speed in a range of 50 to 1200 rpm, at a temperature in the range of 20 to 60° C. In an example, Sample 1 denoted as A_89.8 was prepared by contacting 89.8% (w/w) of MCT with 10% (w/w) of isopropyl myristate and 0.2% (w/w) of vitamin E under stirring at a speed of 150 rpm, at a temperature of 27° C. for a time period in a range of 10 to 60 minutes.

TABLE 1

| | | Ester of | |
|---|---|---|---|
| | MCT % | fatty acid | Vitamin E % |
| Samples | (w/w) | % (w/w) | (w/w) |
| Sample 1 | 89.8 | 10 (isopropyl | 0.2 |
| (A_89.8) | | myristate) | |
| Sample 2 | 89.8 | 10(coco | 0.2 |
| (B_89.8) | | caprylate | |
| | | caprate) | |
| Sample 3 | 74.8 | 25(isopropyl | 0.2 |
| (A_74.8) | | myristate) | |
| Sample 4 | 74.8 | 25(coco | 0.2 |
| (B_74.8) | | caprylate | |
| | | caprate) | |
| Sample 5 | 59.8 | 40(isopropyl | 0.2 |
| (A_59.8) | | myristate) | |
| Sample 6 | 59.8 | 40(coco | 0.2 |
| (B_59.8) | | caprylate | |
| | | caprate) | |

### Sensorial Analysis

The samples prepared as mentioned in Table 1 were further evaluated to analyze their sensorial effects. For this, a panel of 9 healthy volunteers, aged between 25 to 50 having training in sensorial evaluation were selected and subjected to an exhaustive series of testing parameters to determine the sensorial efficacy of the samples for topical composition against the commercial silicones (Cyclopentasiloxane, Dimethicone). Volunteers were asked to first analyze each sample in terms of appearance and smell and then, to apply the sample on forearm in circular movements until disappearance. To evaluate the acceptability of the topical compositions, a questionnaire was answered by each volunteer to assess sensory parameters such as fragrance, texture, initial skin feel, shine, absorption, skin feel on application, after feel, and effect on the skin. The volunteers were required to assign scores using a scale from 0 to 5 where 5 is the highest score on scale. For instance, in skin feel on application, the score of 5 implies a smoother feel and 0 implies a rough feel.

### Moisturization Analysis

The topical composition samples of the present disclosure and the commercial silicones (cyclopentasiloxane, dimethicone) were applied on a skin patch and the moisture content was analyzed for a duration of 8 hrs. The test was conducted on 3 volunteers. Three areas were marked on the skin of the hand. The composition was applied by rubbing in a circular direction for a period of two minute on the marked areas. The third area was kept blank. The readings were taken after five minutes of drying by Scaler Moisture Checker. The temperature of the room was kept at 20° C. and humidity was maintained at 60% RH. The moisture analysis was done using moisture checker from Scalar Impressive works. Scalar moisture checker works on the principle of capacitance to measure the permittivity of skin. The readings between 27-29 showed normal skin type. The checker was pressed on the desired area perpendicularly to the skin and was held for a few seconds. The checker displayed the amount of moisture on the surface at baseline, after application, and at 2, 4, 6, 8 hours post application. Moisture level Content of 45.1 and above represent a very high moisture level, when moisture level is between 39.1-45.0 it is high normal, between 27.1-39.0 is mid normal, between 22.1-27.0 is low normal, between 12.1 to 22.0 is low and below 12.1 is very low moisture level. A graph of time after application and the moisture level content was plotted for each composition.

### Skin Reactivity Analysis

Skin reactivity analysis was carried out for the topical composition samples by agar diffusion assay. Murine fibroblast cell line (3T3) cells were seeded onto 60 mm tissue culture grade plastic Petri dishes and incubated for 24 hrs. After 24 hrs the media was replaced by a layer of 1% agar. Stock solution of 100 mg/ml of reference standard was prepared in sterile phosphate buffered saline (PBS) and tested at 10% (100 mg/ml). 5 μl of reference standard was loaded on to 6 mm sterile Whatman filter paper discs which were placed on the agar layer. Neat topical composition sample was used for testing. Approximately 5 μl of the topical composition sample was loaded on to 6 mm sterile Whatman filter paper discs which were placed on the agar layer. Sterile PBS was used as the negative control. Positive and negative controls were run concurrently. After an exposure of 24 hours to the samples, the assays were terminated by the addition of MTT (3-[4,5-dimethylthiazol-2-yl]-2,5-diphenyltetrazolium bromide; Thiazolyl blue) (Sigma M 5655) and incubated overnight in the dark. The following day, the cells were observed under the microscope and checked for the presence of crystals under the area of sample loading. The sample causing cell death was indicated by a clear zone of inhibition. The colour was allowed to develop overnight and the zone of inhibition was measured. The readings of the test samples were then scored based on comparisons with the positive and negative controls in compliance with the USP (U.S. Pharmacopoeia) grades for the agar diffusion test (Table 2).

TABLE 2

| | | Description of |
|---|---|---|
| Grade | Reactivity | Reactivity Zone |
| 0 | None | No detectable zone around |
| | | or under specimen |
| 1 | Slight | Some malformed or degenerated |
| | | cells under specimen |
| 2 | Mild | Zone limited to area under |
| | | specimen |
| 3 | Moderate | Zone extends 0.5 to 1.0 cm |
| | | beyond specimen |
| 4 | Severe | Zone extends greater than |
| | | 1.0 cm beyond specimen |

### Stability Analysis

The stability of the samples was analysed using accelerated stability test, which was performed at 45° C. for a duration of 3 months and at 70% RH.

### Cytocompatibility Analysis

The cytocompatibility of the samples was assessed using sulforhodamine B (SRB) assay. Proliferation assessment of samples on murine fibroblast cell line (3T3) by Sulforhodamine B (SRB) assay was designed to study the cytotoxicity potential of the topical composition samples in terms of the inhibition of cell growth in comparison with the internal reference standard. Stock solution of 100 mg/ml of reference standard was prepared in sterile phosphate buffered saline (PBS). All concentrations were prepared by serial dilutions and were freshly prepared just before use in phosphate buffered saline. The topical composition sample was tested at concentrations of 0.5%, 1%, 2.5% and 5%. The stock solution was freshly prepared in dimethylsulfoxide (DMSO) and vortexed. Further dilutions were prepared in DMSO. Vehicle controls were run concurrently. Higher concentrations were limited by solubility. The cells were seeded into a 96-well plate and incubated for 24 hrs. After 24 hrs the test compounds were added to the respective wells in quadruplicates and incubated for 24 hrs. After 24 hrs the cells were fixed, washed and dried. The cells were stained with Sulforhodamine B stain, washed, dried and the stain was solubilized in Tris (tris(hydroxymethyl)aminomethane). The plate was read at 570 nm using a plate reader. The intensity of the color was directly proportional to the total protein, which is an indication of cell viability. The cytotoxicity/proliferation is scored based on comparison with the untreated control. Reference standards were used to indicate cytotoxicity and/or proliferation. The readings of the test samples were scored based on comparisons with the zero control (baseline). Optical density of the zero control is considered as 100% viability. Percent viability of the samples were calculated in comparison with the control. IC₅₀ values from dose response curves were also obtained.

Percent Viability=t/c×100

where, t—optical density of sample; c—optical density of the zero control.

### Gloss Test Analysis

The ability of the composition to provide glossiness to hair post its application was assessed using the gloss test. The refractive index of the hair-care composition was evaluated using Gloss Meter. The amount of light reflected from the surface of an object is used to measure the gloss level on the surface. Herein, a hair swatch was placed under the lens of the gloss meter pre and post application of the sample and the gloss meter was operated at 20, 60, 85 degree angles, and the corresponding readings were recorded and analysed.

### Example 2

### Sensorial Analysis: Sample 1(A_89.8)

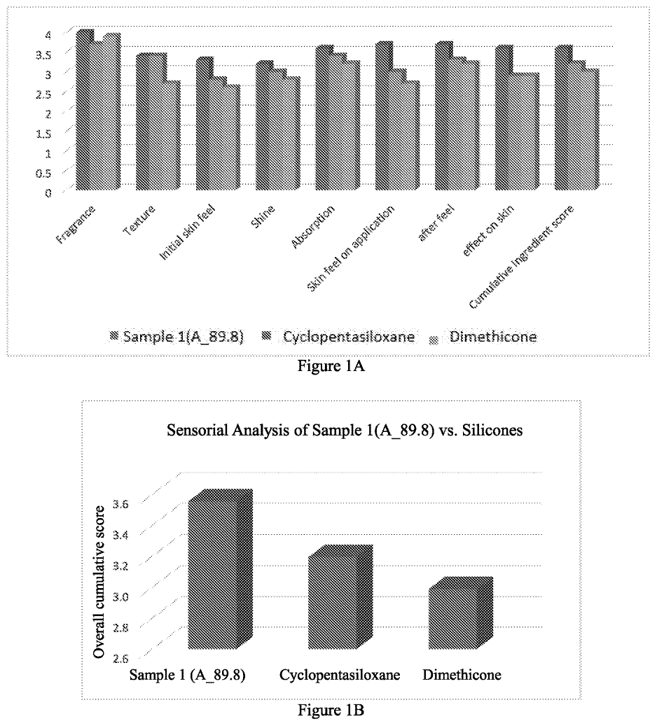

The present example explains the sensory evaluation of the sample 1(A_89.8) comprising 89.8% MCT, 10% isopropyl myristate and 0.2% vitamin E against the commercial silicones (Cyclopentasiloxane, Dimethicone). The sensorial analysis plays an essential role in understanding the acceptance of the product by the consumer. The scores assigned by each volunteer to assess sensory parameters such as fragrance, texture, initial skin feel, shine, absorption, skin feel on application, after feel, and effect on the skin for the samples were tabulated and analysed, to arrive at a consolidated rating for every individual category (Table 3 and FIG. 1A).

The recorded individual scores of the nine volunteers for the above said parameters were tabulated and averaged, to arrive at a consolidated rating for every individual parameter (cumulative score). Then, the overall cumulative score of all the parameters for each sample was obtained as an average of each parameter's consolidated score.

TABLE 3

| | | | | | | | | | | | | Overall |
|---|---|---|---|---|---|---|---|---|---|---|---|---|
| | | | | | | | | | | | Cumulative | Cumulative |
| Sample | Parameter | Volunteer responses | | | | | | | | | Score | score |
| Sample 1 | Fragrance | 5 | 5 | 0 | 4 | 5 | 5 | 3 | 5 | 4 | 4.0 | 3.6 |
| (A_89.8) | Texture | 3 | 3 | 3 | 3 | 5 | 4 | 3 | 4 | 3 | 3.4 | |
| | Initial | 2 | 2 | 4 | 4 | 4 | 4 | 3 | 4 | 3 | 3.3 | |
| | Skin Feel | | | | | | | | | | | |
| | Shine | 4 | 2 | 4 | 3 | 2 | 4 | 3 | 4 | 3 | 3.2 | |
| | Absorption | 3 | 3 | 4 | 4 | 4 | 3 | 4 | 3 | 4 | 3.6 | |
| | Skin feel on | 3 | 4 | 5 | 4 | 4 | 3 | 3 | 3 | 4 | 3.7 | |
| | application | | | | | | | | | | | |
| | After feel | 2 | 4 | 5 | 4 | 5 | 3 | 3 | 3 | 4 | 3.7 | |
| | Effect | 4 | 1 | 5 | 4 | 4 | 4 | 3 | 3 | 4 | 3.6 | |
| | on skin | | | | | | | | | | | |
| Cyclopenta- | Fragrance | 5 | 5 | 0 | 3 | 5 | 5 | 3 | 5 | 2 | 3.7 | 3.2 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3.0 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

The above scores from the volunteers confirmed that the sample 1(A_89.8) comprising 89.8% MCT, 10% isopropyl myristate and 0.2% vitamin E provided better sensorial effects as shown in Table 2. The sample 1(A_89.8) had the highest overall cumulative score of 3.6 as against the silicones with 3.2 for cyclopentasiloxanes and 3.0 for dimethicone (Table 3 & FIG. 1B).

### Moisturization Analysis: a Comparison Between Sample 1(A_89.8) Against Silicones (Cyclopentasiloxane, Dimethicone)

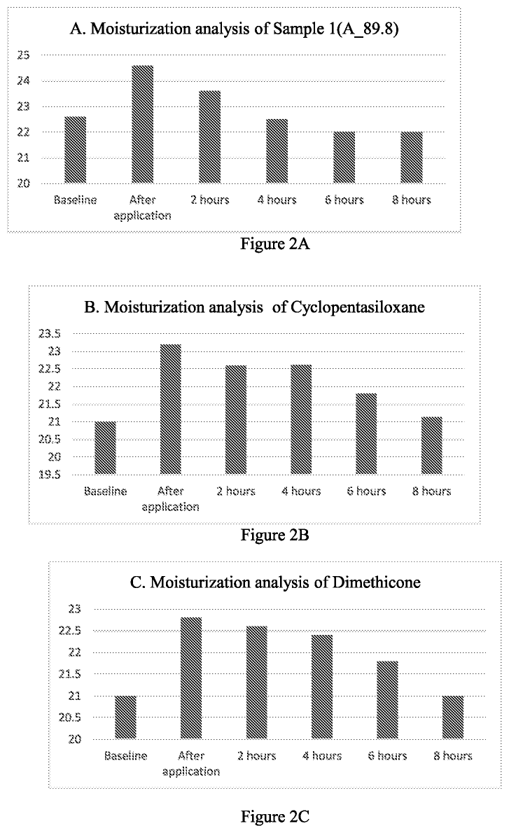

Moisturization analysis of the sample 1(A_89.8) was evaluated against commercial silicones cyclopentasiloxane, and dimethicone. It is evident from FIG. 2 (A, B and C) that the sample 1(A_89.8) showed 6-7% enhanced moisturization abilities than cyclopentasiloxane and dimethicone. Sample 1 (A_89.8) exhibited moisture content of 24.5% of moisture content after application and is retained up to 22% until 8 hours after application. Whereas cyclopentasiloxane and dimethicone exhibited moisture content of 23%, 22.5% respectively after application and is retained up to 21% each until 8 hours after application. Thus, it is clear that the topical composition of the present disclosure sample 1(A_89.8) exhibited improved moisturization effects on skin even after 8 hours of application.

Stability of the Sample 1(A_89.8) was determined under 70% RH at 45° C. and it was found that the composition was stable for at least 3 months under said conditions.

### Skin Reactivity Analysis of Sample 1(A_89.8)

Skin reactivity of Sample 1(A_89.8) along with positive and negative control samples were assessed by agar diffusion test. The objective of the agar diffusion assay was to study the reactivity by determining the inhibition zone due to the test samples on Murine Fibroblast cell line (3T3). A total of 3 independent experiments were carried out. Reactivity of the samples were assessed by the measurement of the inhibition zone. The assay was carried out to study the effect of the sample for 24 hrs, using the murine fibroblast cell line (3T3). The zones of inhibition obtained for Sample 1(A_89.8) for three independent experiments along with positive and negative control is presented in Table 4. Further, the mean of three independent experiments is presented in Table 5. The readings of the samples were then scored based on comparisons with the positive and negative controls in compliance with the USP grades for Agar Diffusion Test as shown in Table 6.

TABLE 4
Zone of Inhibition (cm)

| | Sample | Positive | Negative |
|---|---|---|---|
| | 1(A_89.8) | Control | Control |
| Expt 1 | 0.00 | 1.20 | 0.00 |
| | 0.00 | 1.18 | 0.00 |
| | 0.00 | 1.22 | 0.00 |
| Mean | 0.00 | 1.20 | 0.00 |
| Expt 2 | 0.00 | 1.05 | 0.00 |
| | 0.00 | 1.03 | 0.00 |
| | 0.00 | 1.17 | 0.00 |
| Mean | 0.00 | 1.08 | 0.00 |
| Expt 3 | 0.00 | 1.13 | 0.00 |
| | 0.00 | 1.07 | 0.00 |
| | 0.00 | 1.23 | 0.00 |
| Mean | 0.00 | 1.14 | 0.00 |

TABLE 5
Zone of Inhibition (cm)

| | Sample | Positive | Negative |
|---|---|---|---|
| | 1(A_89.8) | Control | Control |
| Expt 1 | 0.00 | 1.20 | 0.00 |
| Expt 2 | 0.00 | 1.08 | 0.00 |
| Expt 3 | 0.00 | 1.14 | 0.00 |
| Mean ± SD | 0.00 | 1.14 ± 0.06 | 0.00 |

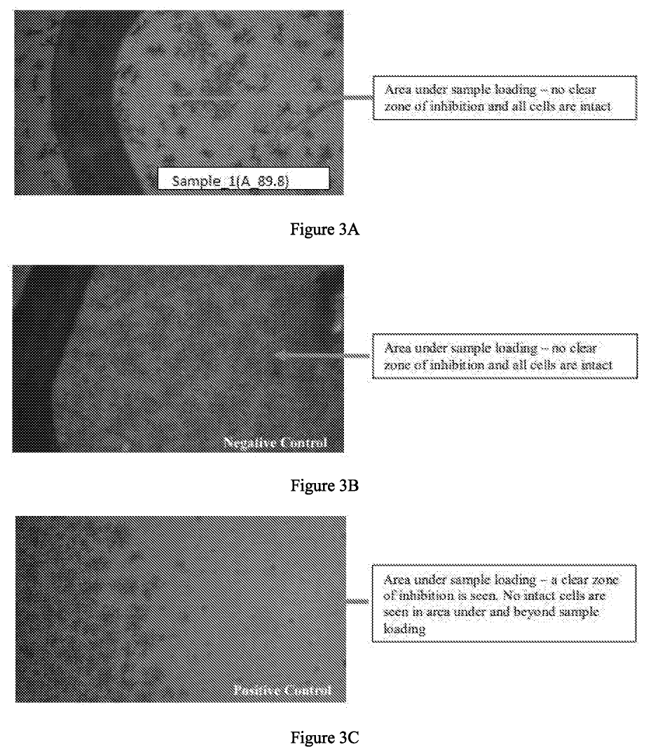

Sample 1(A_89.8) was found to have no zone of inhibition at 100% concentration (Table 4). The positive control was found to have an average zone of inhibition of 1.14±0.06 cm (Table 5). The negative control was found to have no zone of inhibition. Sample 1(A_89.8) was scored under the experimental conditions as shown in Table 6 and was found that the cells under the loading were intact for Sample 1(A_89.8) and not malformed or degenerated (FIG. 3A) when compared to positive control (FIG. 3C); hence sample 1(A_89.8) was found to be non-reactive and compatible for skin cells.

TABLE 6

| | 1 | 2 | 3 |
|---|---|---|---|
| S. No. | Sample | Positive | Negative |
| Sample | 1(A_89.8) | Control | Control |
| Grade | 0 | 4 | 0 |
| Reactivity | None | Severe | None |
| Description of | No detectable | Zone extends | No detectable |
| Reactivity Zone | zone around | greater than | zone around |
| | or under | 1.0 cm beyond | or under |
| | specimen | specimen | specimen |

### Cytocompatibility Analysis of Sample 1(A_89.8)

Cytocompatibility of Sample 1(A_89.8) was assessed by the determination of its cytotoxicity/proliferation using the Sulforhodamine B Assay. A total of 3 independent experiments per sample were carried out. The ability of the samples to induce cytotoxicity were assessed by the Sulforhodamine B (SRB) method. The assay was carried out to study the effect of the sample for 24 hrs, using the murine fibroblast cell line (3T3). A total of four concentrations ranging from 0.5% to 5% were used for the study. Concentrations ranging from 0.005% to 0.02% were used for the reference standard. Percentage viabilities were calculated for the topical composition and the reference standard. Table 7 shows the individual percentage viability for each experiment for different concentration for both sample 1 and the reference standard. Mean values of the percentage viability of the reference standard and sample 1 are also tabulated (Table 8).

TABLE 7

| Sample | Percentage Viability | | | |
|---|---|---|---|---|
| Sample | 0.5% | 1% | 2.5% | 5% |
| 1(A_89.8) | (5 mg/ml) | (10 mg/ml) | (25 mg/ml) | (50 mg/ml) |
| Expt 1 | 75.73 | 71.84 | 87.06 | 100 |
| Expt 2 | 72.17 | 66.99 | 80.26 | 99.35 |
| Expt 3 | 72.17 | 65.05 | 82.2 | 83.82 |
| Mean | 73.35 | 67.96 | 83.17 | 94.39 |
| Standard | ±2.06 | ±3.5 | ±3.5 | ±9.16 |
| Deviation | | | | |
| (SD) | | | | |

| Sample | Percentage Viability | | | |
|---|---|---|---|---|
| Reference | 0.005% | 0.01% | 0.015% | 0.02% |
| Standard | (0.05 mg/ml) | (0.1 mg/ml) | (0.15 mg/ml) | (0.2 mg/ml) |
| Expt 1 | 91.78 | 62.01 | 28.95 | 16.35 |
| Expt 2 | 87.85 | 50.87 | 50.87 | 50.87 |
| Expt 3 | 91.56 | 73.54 | 29.53 | 20.23 |
| Mean | 90.4 | 62.14 | 26.49 | 20.34 |
| Standard | ±2.21 | ±11.34 | ±4.77 | ±4.05 |
| Deviation | | | | |
| (SD) | | | | |

TABLE 8

| | Mean viability at different concentration | | | |
|---|---|---|---|---|
| Sample | 0.5% | 1% | 2.5% | 5% |
| Concentration | (5 mg/ml) | (10 mg/ml) | (25 mg/ml) | (50 mg/ml) |
| Sample | 73.35 ± 2.06 | 67.96 ± 3.5 | 83.17 ± 3.5 | 94.39 ± 9.16 |
| 1(A_89.8) | | | | |

| | 0.005% | 0.01% | 0.015% | 0.02% |
|---|---|---|---|---|
| Concentration | (0.05 mg/ml) | (0.1 mg/ml) | (0.15 mg/ml) | (0.2 mg/ml) |
| Reference | 90.4% ± 2.21 | 62.14% ± 11.34 | 26.49% ± 4.77 | 20.34% ± 4.05 |
| Standard | | | | |

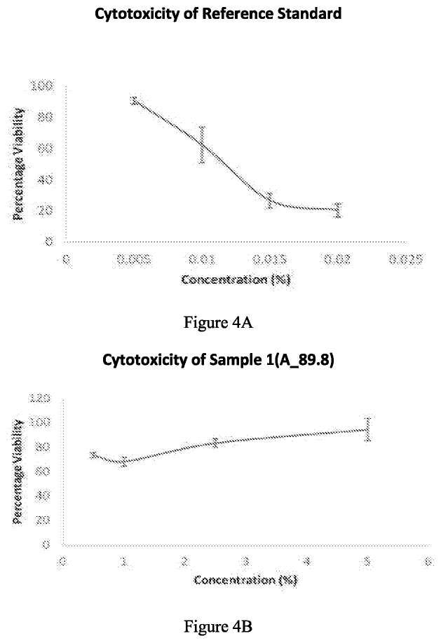

Reference standard was seen to have a maximum viability of 90.40%±2.21 at 0.005%, which decreased in a dose dependent manner to 20.34%±4.05 at 0.02% (Table 8 and FIG. 4A). IC₅₀ value for reference standard was determined to be 0.0115%. Sample 1(A_89.8) on the other hand induced a cell viability of 73.35%±2.06 at the lowest concentration of 0.5% (5 mg/ml) and 94.39%±9.16 at the highest concentration of 5% (50 mg/ml) (Table 8 and FIG. 4B). There was no dose dependent cytotoxicity and the IC₅₀ value could not be determined. It can be understood that the sample 1(A_89.8) is not cytotoxic and is cytocompatible.

### Example 3

### Sensorial Analysis Sample 2 (B_89.8)

Sample 2 (B_89.8) comprising 89.8% MCT, 10% coco caprylate caprate and 0.2% vitamin E prepared as explained in Example 1 was subjected to sensorial evaluation against commercial silicones (Cyclopentasiloxane, Dimethicone) and scores recorded by the volunteers were consolidated. The scores assigned by each volunteer to assess sensory parameters such as fragrance, texture, initial skin feel, shine, absorption, skin feel on application, after feel, and effect on the skin for the samples were tabulated and analyzed, to arrive at a consolidated rating for every individual category (Table 9).

The recorded individual scores of the nine volunteers for the parameters were tabulated and averaged, to arrive at a consolidated rating (cumulative score) for every individual parameter. Then, the overall cumulative score of all the parameters for each sample was obtained as an average of each parameter's consolidated score.

TABLE 9

| | | | | | | | | | | | | Overall |
|---|---|---|---|---|---|---|---|---|---|---|---|---|
| | | Volunteer Responses | | | | | | | | | Cumulative | Cumulative |
| Sample | Parameter | 1 | 2 | 3 | 4 | 5 | 6 | 7 | 8 | 9 | score | Score |
| Sample 2 | Fragrance | 5 | 5 | 3 | 3 | 5 | 5 | 2 | 1 | 5 | 3.8 | 3.4 |
| (B_89.8) | Texture | 2 | 4 | 4 | 3 | 4 | 3 | 3 | 4 | 3 | 3.3 | |
| | Initial | 3 | 3 | 2 | 2 | 2 | 3 | 2 | 3 | 3 | 2.6 | |
| | Skin Feel | | | | | | | | | | | |
| | Shine | 3 | 3 | 2 | 3 | 5 | 3 | 3 | 3 | 3 | 3.1 | |
| | Absorption | 4 | 4 | 4 | 4 | 5 | 4 | 4 | 5 | 5 | 4.3 | |
| | Skin feel on | 2 | 4 | 4 | 3 | 4 | 3 | 3 | 4 | 4 | 3.4 | |
| | application | | | | | | | | | | | |
| | After feel | 4 | 3 | 3 | 3 | 5 | 3 | 4 | 4 | 4 | 3.7 | |
| | Effect | 2 | 2 | 3 | 4 | 5 | 3 | 3 | 3 | 2 | 3.0 | |
| | on skin | | | | | | | | | | | |
| Cyclopenta- | Fragrance | 5 | 5 | 0 | 3 | 5 | 5 | 3 | 5 | 2 | 3.7 | 3.2 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3.0 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

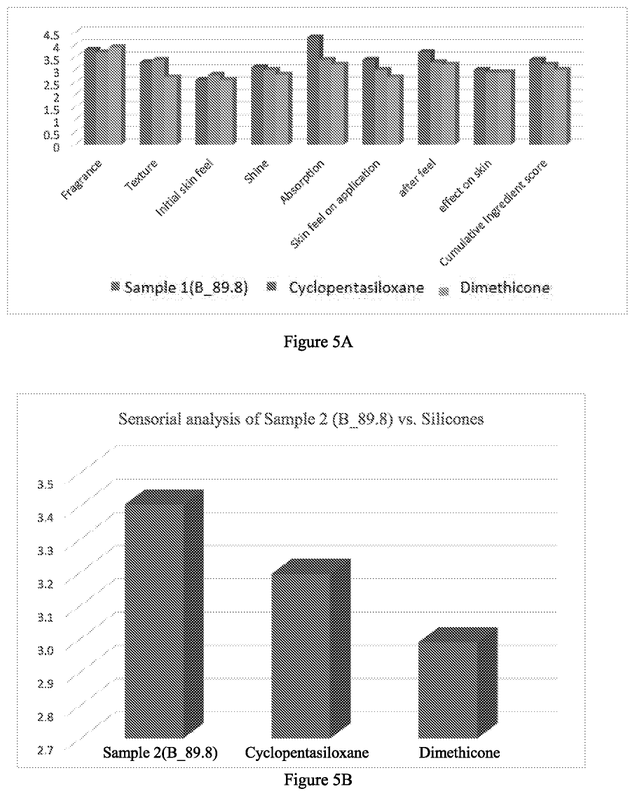

The scores from the volunteers confirmed that the Sample 2 (B_89.8) comprising 89.8% MCT, 10% coco caprylate caprate and 0.2% vitamin E provided better sensorial effect in the parameters as shown in Table 9 and FIG. 5A. Sample 2 (B_89.8) had the highest overall cumulative score of 3.4 as against the silicones with 3.2 for cyclopentasiloxanes and 3.0 for dimethicone (Table 9 & FIG. 5B).

Stability of the Sample 2 (B_89.8) was determined under 70% RH at 45° C. and it was found that the composition was stable for at least 3 months under said conditions.

### Moisturization Analysis: a Comparison Between Sample 2 (B_89.8) Against Silicones (Cyclopentasiloxane, Dimethicone)

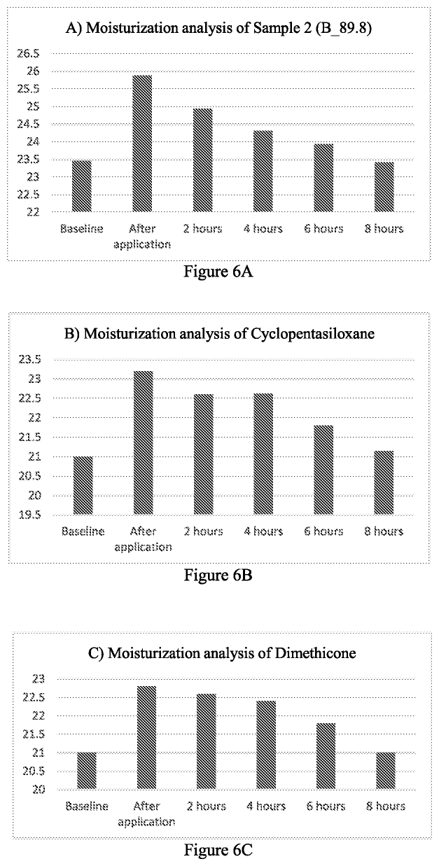

Moisturization analysis of the Sample 2 (B_89.8) was evaluated against commercial silicones—Cyclopentasiloxane, Dimethicone. It is evident from FIG. 6 (A, B and C) that the Sample 2 (B_89.8) showed 7-8% enhanced moisturization abilities than cyclopentasiloxane and dimethicone.

### Skin Reactivity Analysis of Sample 2(B_89.8)

Skin reactivity of Sample 2(B_89.8) along with positive and negative control samples was assessed by Agar Diffusion test. The agar diffusion assay was used to study the reactivity by determining the inhibition zone due to the samples on murine fibroblast cell line (3T3). A total of 3 independent experiments were carried out. Reactivity of the samples were assessed by the measurement of the inhibition zone. The zones of inhibition obtained for Sample 2(B_89.8) for three independent experiments along with positive and negative control is shown in Table 10. Further, the mean of three independent experiments is presented in Table 11. The readings of the samples were then scored based on comparisons with the positive and negative controls in compliance with the USP grades for agar diffusion test as shown in Table 12.

TABLE 10
Zone of Inhibition (cm)

| | Sample | Positive | Negative |
|---|---|---|---|
| | 2(B_89.8) | Control | Control |
| Expt 1 | 0.00 | 1.20 | 0.00 |
| | 0.00 | 1.18 | 0.00 |
| | 0.00 | 1.22 | 0.00 |
| Mean | 0.00 | 1.20 | 0.00 |
| Expt 2 | 0.00 | 1.05 | 0.00 |
| | 0.00 | 1.03 | 0.00 |
| | 0.00 | 1.17 | 0.00 |
| Mean | 0.00 | 1.08 | 0.00 |
| Expt 3 | 0.00 | 1.13 | 0.00 |
| | 0.00 | 1.07 | 0.00 |
| | 0.00 | 1.23 | 0.00 |
| Mean | 0.00 | 1.14 | 0.00 |

TABLE 11
Zone of Inhibition (cm)

| | Sample | Positive | Negative |
|---|---|---|---|
| | 2(B_89.8) | Control | Control |
| Expt 1 | 0.00 | 1.20 | 0.00 |
| Expt 2 | 0.00 | 1.08 | 0.00 |
| Expt 3 | 0.00 | 1.14 | 0.00 |
| Mean ± SD | 0.00 | 1.14 ± 0.06 | 0.00 |

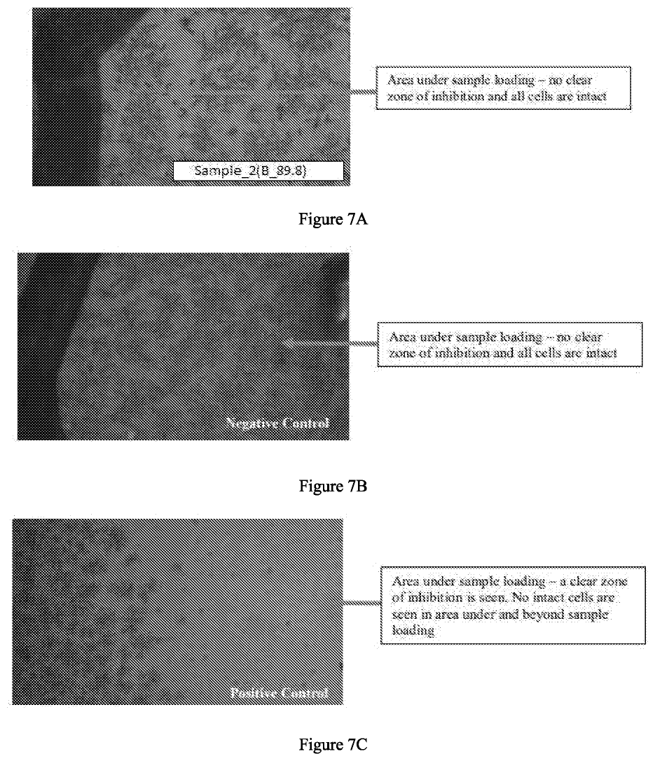

Sample 2(B_89.8) was found to have no zone of inhibition at 100% concentration (Table 10). The positive control was found to have an average zone of inhibition of 1.14±0.06 cm (Table 11). The negative control was found to have no zone of inhibition. As per the guidelines prescribed by the U.S. Pharmacopoeia, Biological tests, the Sample 2(B_89.8) was scored under the experimental conditions as shown in Table 12. Further, the cells under the loading were found to be intact for Sample 2(B_89.8) and not malformed or degenerated (FIG. 7A), when compared to negative and positive controls (FIGS. 7B and C); hence sample 2(B_89.8) was found to be non-reactive.

TABLE 12

| | 1 | 2 | 3 |
|---|---|---|---|
| S. No. | Sample | Positive | Negative |
| Sample | 2(B_89.8) | Control | Control |
| Grade | 0 | 4 | 0 |
| Reactivity | None | Severe | None |
| Description of | No detectable | Zone extends | No detectable |
| Reactivity Zone | zone around | greater than | zone around |
| | or under | 1.0 cm beyond | or under |
| | specimen | specimen | specimen |

### Cytocompatibility Analysis of Sample 2(B_89.8)

Cytocompatibility of Sample 2(B_89.8) was assessed by the determination of its cytotoxicity/proliferation using the Sulforhodamine B Assay. A total of 3 independent experiments were carried out. The ability of the samples to induce cytotoxicity were assessed by the Sulforhodamine B (SRB) method. The assays were carried out to study the effect of the sample for 24 hrs, using the murine fibroblast cell line (3T3). A total of four concentrations ranging from 0.5% to 5% were used for the sample 2 (B_89.8) depending on solubility. Concentrations ranging from 0.005% to 0.02% were used for the reference standard. A summary of the percentage viability determined is shown in Table 13.

TABLE 13

| Sample | Percentage Viability | | | |
|---|---|---|---|---|
| Sample | 0.5% | 1% | 2.5% | 5% |
| 2(B_89.8) | (5 mg/ml) | (10 mg/ml) | (25 mg/ml) | (50 mg/ml) |
| Expt 1 | 71.84 | 59.22 | 71.84 | 79.61 |
| Expt 2 | 66.99 | 55.99 | 71.52 | 84.79 |
| Expt 3 | 66.99 | 58.9 | 71.2 | 77.99 |
| Mean | 68.61 | 58.04 | 71.52 | 80.80 |
| Standard | ±2.8 | ±3.5 | ±1.78 | ±3.55 |
| Deviation (SD) | | | | |

| Sample | Percentage Viability | | | |
|---|---|---|---|---|
| Reference | 0.005% | 0.01% | 0.015% | 0.02% |
| Standard | (0.05 mg/ml) | (0.1 mg/ml) | (0.15 mg/ml) | (0.2 mg/ml) |
| Expt 1 | 91.78 | 62.01 | 28.95 | 16.35 |
| Expt 2 | 87.85 | 50.87 | 50.87 | 50.87 |
| Expt 3 | 91.56 | 73.54 | 29.53 | 20.23 |
| Mean | 90.4 | 62.14 | 26.49 | 20.34 |
| Standard | ±2.21 | ±11.34 | ±4.77 | ±4.05 |
| Deviation (SD) | | | | |

TABLE 14

| | Mean viability at different concentration | | | |
|---|---|---|---|---|
| Sample | 0.5% | 1% | 2.5% | 5% |
| Concentration | (5 mg/ml) | (10 mg/ml) | (25 mg/ml) | (50 mg/ml) |
| Sample | 68.61 ± 2.8 | 58.04 ± 3.5 | 71.52 ± 1.78 | 80.80 ± 3.55 |
| 2(B_89.8) | | | | |

| | Percentage Viability | | | |
|---|---|---|---|---|
| Sample | 0.005% | 0.01% | 0.015% | 0.02% |
| Concentration | (0.05 mg/ml) | (0.1 mg/ml) | (0.15 mg/ml) | (0.2 mg/ml) |
| Reference | 90.4% ± 2.21 | 62.14% ± 11.34 | 26.49% ± 4.77 | 20.34% ± 4.05 |
| Standard | | | | |

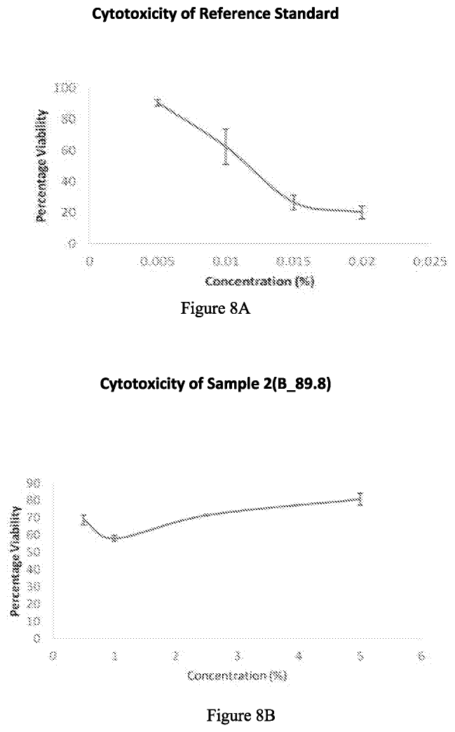

Reference standard was seen to have a maximum viability of 90.40%±2.21 at 0.005%, which reduced in a dose dependent manner to 20.34%±4.05 at 0.02% (Table 14 and FIG. 8A). IC₅₀ value for reference standard was determined to be 0.0115%. Sample 2(B_89.8) induced a cell viability of 68.61%±2.8 at the lowest concentration of 0.5% (5 mg/ml) and 80.80%+3.55 at the highest concentration of 5% (50 mg/ml) (Table 14 and FIG. 8B). There was no dose dependent cytotoxicity and the IC50 value could not be determined. Thus, it can be understood that the Sample 2(B_89.8) is not cytotoxic and is cytocompatible.

### Comparative Example 1

### Sensorial Analysis: Sample 3 (A_74.8)

Sensory evaluation of the Sample 3 (A_74.8) comprising 74.8% MCT, 25% isopropyl myristate and 0.2% vitamin E was recorded and compared against commercial silicones (Cyclopentasiloxane, Dimethicone). The scores assigned by each volunteer to assess sensory parameters such as fragrance, texture, initial skin feel, shine, absorption, skin feel on application, after feel, and effect on the skin for the samples were tabulated and analysed, to arrive at a consolidated rating for every individual category (Table 15).

The recorded individual scores of the nine volunteers for the parameters were tabulated and averaged, to arrive at a consolidated rating (cumulative score) for every individual parameter. Then, the overall cumulative score of all the parameters for each sample was obtained as an average of each parameter's consolidated score.

TABLE 15

| | | | | | | | | | | | | Overall |
|---|---|---|---|---|---|---|---|---|---|---|---|---|
| | | Volunteer responses | | | | | | | | | Cumulative | cumulative |
| Sample | Parameter | 1 | 2 | 3 | 4 | 5 | 6 | 7 | 8 | 9 | score | score |
| Sample 3 | Fragrance | 3 | 2 | 1 | 2 | 3 | 4 | 3 | 3 | 2 | 2.6 | 2.9 |
| (A_74.8) | Texture | 2 | 3 | 3 | 2 | 4 | 4 | 3 | 2 | 2 | 2.8 | |
| | Initial | 2 | 3 | 3 | 4 | 3 | 3 | 2 | 4 | 3 | 3.0 | |
| | Skin Feel | | | | | | | | | | | |
| | Shine | 3 | 4 | 2 | 3 | 3 | 2 | 3 | 4 | 4 | 3.1 | |
| | Absorption | 4 | 2 | 4 | 2 | 2 | 4 | 3 | 2 | 4 | 3.0 | |
| | Skin feel on | 2 | 2 | 3 | 4 | 3 | 4 | 3 | 2 | 2 | 2.8 | |
| | application | | | | | | | | | | | |
| | After feel | 3 | 3 | 3 | 3 | 4 | 3 | 3 | 3 | 3 | 3.1 | |
| | Effect | 3 | 4 | 3 | 2 | 4 | 3 | 1 | 3 | 4 | 3.0 | |
| | on skin | | | | | | | | | | | |
| Cyclopenta- | Fragrance | 5 | 5 | 0 | 3 | 5 | 5 | 3 | 5 | 2 | 3.7 | 3.2 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

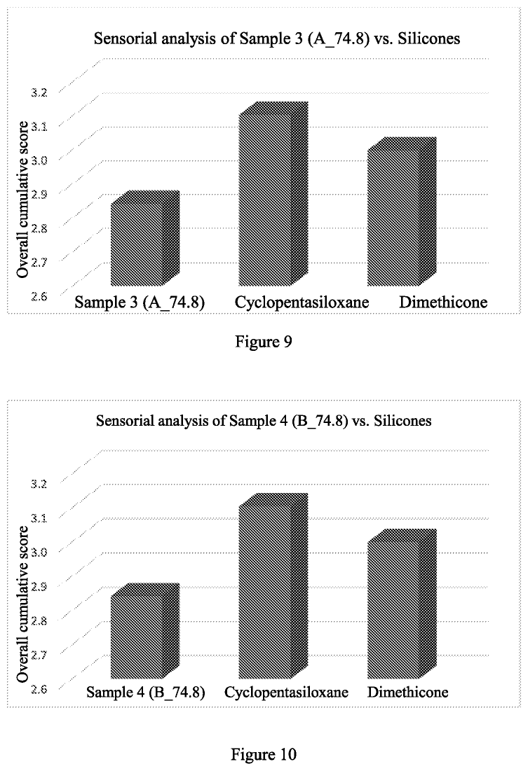

From Table 15 it can be observed that the silicones were found to be more acceptable than the Sample 3 (A_74.8) comprising 74.8% MCT, 25% isopropyl myristate and 0.2% vitamin E in the sensory parameters. Sample 3 (A_74.8) had the least overall cumulative score of 2.9 as against commercial silicones with 3.2 for cyclopentasiloxanes and 3.0 for dimethicone (FIG. 9). This decrease in the sensorial effect could be attributed to the % (w/w) of isopropyl myristate used in this composition. It can be seen that the weight % of isopropyl myristate is higher than the specified weight % range of 1 to 15% and confirmed that the increase in weight % of isopropyl myristate resulted in a composition of undesired effect. Thus, it is essential that the components are taken within said weight ranges for the composition to provide enhanced sensorial effect.

### Moisturization Analysis: Sample 3 (A_74.8)

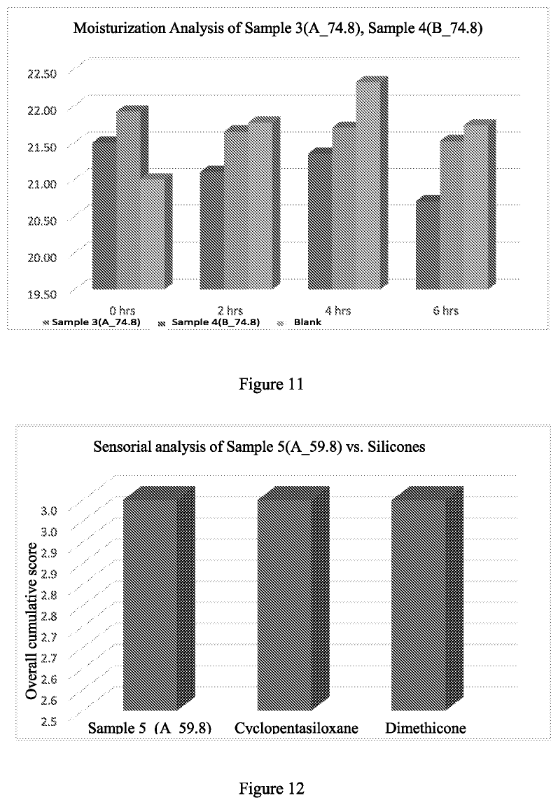

Moisturization analysis of the Sample 3 (A_74.8) was evaluated and it was evident from FIG. 11 that the sample 3(A_74.8) exhibited decreasing moisturization ability post two hours of the test. As can be seen, the composition prepared outside said weight ranges did not provide improved moisturization ability, and instead exhibited declined properties. Thus, it can be confirmed that the composition prepared from said weight ranges as disclosed herein are suitable for a topical composition of enhanced cosmetic applicability.

### Comparative Example 2

### Sensorial Analysis: Sample 4 (B 74.8)

Sensory evaluation of the Sample 4 (B_74.8) comprising 74.8% MCT, 25% coco caprylate caprate and 0.2% vitamin E against commercial silicones (Cyclopentasiloxane, Dimethicone). The scores assigned by each volunteer to assess sensory parameters such as fragrance, texture, initial skin feel, shine, absorption, skin feel on application, after feel, and effect on the skin for the samples were tabulated and analyzed, to arrive at a consolidated rating for every individual category (Table 16).

The recorded individual scores of the nine volunteers for the parameters were tabulated and averaged, to arrive at a consolidated (cumulative score) rating for every individual parameter. Then, the overall cumulative score of all the parameters for each sample was obtained as an average of each parameter's consolidated score.

TABLE 16

| | | | | | | | | | | | | Overall |
|---|---|---|---|---|---|---|---|---|---|---|---|---|
| | | Volunteer responses | | | | | | | | | Cumulative | cumulative |
| Sample | Parameter | 1 | 2 | 3 | 4 | 5 | 6 | 7 | 8 | 9 | score | score |
| Sample 4 | Fragrance | 3 | 2 | 3 | 2 | 4 | 5 | 1 | 2 | 2 | 2.7 | 2.9 |
| (B_74.8) | Texture | 2 | 4 | 3 | 2 | 3 | 4 | 3 | 2 | 3 | 2.9 | |
| | Initial | 3 | 3 | 1 | 3 | 2 | 3 | 3 | 3 | 2 | 2.6 | |
| | Skin Feel | | | | | | | | | | | |
| | Shine | 2 | 4 | 2 | 3 | 3 | 4 | 3 | 2 | 3 | 2.9 | |
| | Absorption | 3 | 3 | 2 | 4 | 3 | 4 | 3 | 2 | 3 | 3.0 | |
| | Skin feel on | 3 | 3 | 3 | 4 | 3 | 2 | 4 | 2 | 2 | 2.9 | |
| | application | | | | | | | | | | | |
| | After feel | 2 | 3 | 3 | 4 | 3 | 4 | 3 | 2 | 4 | 3.1 | |
| | Effect | 2 | 3 | 2 | 4 | 3 | 4 | 3 | 2 | 3 | 2.9 | |
| | on skin | | | | | | | | | | | |
| Cyclopenta- | Fragrance | 5 | 5 | 0 | 3 | 5 | 5 | 3 | 5 | 2 | 3.7 | 3.7 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3.2 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

It can be understood from Table 16 that the silicones were found to be more acceptable than the Sample 4 (B_74.8) comprising 74.8% MCT, 25% coco caprylate caprate and 0.2% vitamin E in the sensory attributes; Sample 4 (B_74.8) had the lowest overall cumulative score of 2.9 as against the silicones, with 3.7 for cyclopentasiloxanes and 3.0 for dimethicone (FIG. 10). This deterioration in the sensorial effect is due to the % (w/w) of coco caprylate caprate used in this composition. Coco caprylate caprate is taken beyond the specified weight % range of 1 to 15%, and the composition resulted in undesired effect. Thus, the presence of all the three components in said weight ranges is essential to achieve the enhanced sensorial effect.

### Moisturization Analysis: Sample 4 (B_74.8)

Moisturization analysis of the Sample 4 (B_74.8) was evaluated and from FIG. 11 it is evident that the Sample 4 (B_74.8) showed decreased moisturization abilities post two hours of the test, similar to sample 3 (A_74.8). Thus, the composition prepared outside said weight ranges resulted in a less desirable topical composition, and again confirmed that it is essential to maintain all the three components within said weight ranges as described herein.

### Comparative Example 3

### Sensorial Analysis: Sample 5 (A_59.8)

Sensory evaluation of the Sample 5 (A_59.8) comprising 59.8% MCT, 40% isopropyl myristate and 0.2% vitamin E against the commercial silicones (Cyclopentasiloxane, Dimethicone) was recorded. The scores assigned by each volunteer to assess sensory parameters such as fragrance, texture, initial skin feel, shine, absorption, skin feel on application, after feel, and effect on the skin for the samples were tabulated and analysed, to arrive at a consolidated rating for every individual category (Table 17).

The recorded individual scores of the nine volunteers for the parameters were tabulated and averaged, to arrive at a consolidated rating (cumulative scores) for every individual parameter. Then, the overall cumulative score of all the parameters for each sample was obtained as an average of each parameter's consolidated score.

TABLE 17

| | | | | | | | | | | | | Overall |
|---|---|---|---|---|---|---|---|---|---|---|---|---|
| | | Volunteer responses | | | | | | | | | Cumulative | cumulative |
| Sample | Parameter | 1 | 2 | 3 | 4 | 5 | 6 | 7 | 8 | 9 | score | score |
| Sample 5 | Fragrance | 5 | 2 | 0 | 2 | 5 | 5 | 3 | 3 | 2 | 3.0 | 3.3 |
| (A_59.8) | Texture | 1 | 5 | 3 | 3 | 5 | 4 | 2 | 2 | 4 | 3.2 | |
| | Initial | 2 | 5 | 3 | 3 | 4 | 4 | 2 | 2 | 5 | 3.3 | |
| | Skin Feel | | | | | | | | | | | |
| | Shine | 2 | 5 | 2 | 2 | 4 | 4 | 1 | 5 | 4 | 3.2 | |
| | Absorption | 4 | 4 | 4 | 2 | 3 | 4 | 5 | 1 | 4 | 3.4 | |
| | Skin feel on | 2 | 2 | 4 | 3 | 4 | 4 | 2 | 2 | 2 | 2.8 | |
| | application | | | | | | | | | | | |
| | After feel | 3 | 3 | 3 | 2 | 4 | 5 | 3 | 3 | 4 | 3.3 | |
| | Effect | 4 | 4 | 4 | 2 | 4 | 4 | 1 | 3 | 4 | 3.3 | |
| | on skin | | | | | | | | | | | |
| Cyclopenta- | Fragrance | 5 | 5 | 0 | 3 | 5 | 5 | 3 | 5 | 2 | 3.7 | 3.2 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3.0 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

Sample 5 (A_59.8) comprising 59.8% MCT, 40% isopropyl myristate and 0.2% vitamin E did not have significant sensory abilities when compared to the silicones in all the attributes as shown in Table 17. The Sample 5 (A_59.8) had the overall cumulative score of 3.3 as against the silicones with 3.2 for cyclopentasiloxanes and 3.0 for dimethicone (FIG. 12). The results showed that the sample 5 exhibited comparable sensorial properties however exhibited poor moisturization property compared to blank post 2 hours of application as evident from the FIG. 14 scores. Further it can also be observed that the Sample 5 (A_59.8) have much lower score than Sample 1 (A_89.8) in fragrance, skin feel on application and after feel factors. This deviation in the property is primarily because sample 5 has lesser MCT % and a higher isopropyl myristate than the described weight ranges. Therefore, for a topical composition to impart improved sensorial as well as moisturization properties, it is critical that the components are taken well within said ranges.

### Comparative Example 4

### Sensorial Analysis: Sample 6 (B_74.8)

Sample 6 (B_59.8) comprising 59.8% MCT, 40% coco caprylate caprate and 0.2% vitamin E and commercial silicones (Cyclopentasiloxane, Dimethicone) were subjected to sensory evaluation and the scores assigned by each volunteer to assess sensory parameters such as fragrance, texture, initial skin feel, shine, absorption, skin feel on application, after feel, and effect on the skin for the samples were tabulated and analyzed, to arrive at a consolidated rating for every individual category (Table 18).

The recorded individual scores of the nine volunteers for the parameters were tabulated and averaged, to arrive at a consolidated rating (cumulative score) for every individual parameter. Then, the overall cumulative score of all the parameters for each sample was obtained as an average of each parameter's consolidated score.

TABLE 18

| | | | | | | | | | | | | Overall |
|---|---|---|---|---|---|---|---|---|---|---|---|---|
| | | Volunteer responses | | | | | | | | | Cumulative | cumulative |
| Sample | Parameter | 1 | 2 | 3 | 4 | 5 | 6 | 7 | 8 | 9 | score | score |
| Sample 6 | Fragrance | 4 | 2 | 2 | 2 | 5 | 5 | 1 | 3 | 2 | 2.9 | 3.4 |
| (B_59.8) | Texture | 2 | 4 | 4 | 3 | 4 | 5 | 3 | 4 | 4 | 3.7 | |
| | Initial | 2 | 4 | 2 | 3 | 5 | 5 | 2 | 3 | 4 | 3.3 | |
| | Skin Feel | | | | | | | | | | | |
| | Shine | 3 | 4 | 1 | 3 | 4 | 5 | 2 | 5 | 4 | 3.4 | |
| | Absorption | 2 | 4 | 2 | 3 | 3 | 5 | 5 | 4 | 3 | 3.4 | |
| | Skin feel on | 3 | 3 | 4 | 3 | 5 | 5 | 3 | 3 | 2 | 3.4 | |
| | application | | | | | | | | | | | |
| | After feel | 2 | 4 | 3 | 3 | 4 | 5 | 3 | 3 | 5 | 3.6 | |
| | Effect | 2 | 2 | 2 | 3 | 3 | 5 | 3 | 4 | 3 | 3.0 | |
| | on skin | | | | | | | | | | | |
| Cyclopenta- | Fragrance | 5 | 5 | 0 | 3 | 5 | 5 | 3 | 5 | 2 | 3.7 | 3.2 |
| siloxane | Texture | 3 | 4 | 4 | 4 | 3 | 4 | 3 | 4 | 2 | 3.4 | |
| | Initial | 4 | 4 | 2 | 3 | 2 | 3 | 3 | 3 | 1 | 2.8 | |
| | Skin Feel | | | | | | | | | | | |
| | Shine | 4 | 4 | 2 | 4 | 2 | 3 | 2 | 3 | 3 | 3.0 | |
| | Absorption | 4 | 3 | 3 | 3 | 3 | 3 | 5 | 4 | 3 | 3.4 | |
| | Skin feel on | 4 | 2 | 3 | 3 | 3 | 4 | 3 | 3 | 2 | 3.0 | |
| | application | | | | | | | | | | | |
| | After feel | 3 | 3 | 4 | 4 | 3 | 4 | 3 | 4 | 2 | 3.3 | |
| | Effect | 4 | 3 | 4 | 4 | 3 | 3 | 2 | 1 | 2 | 2.9 | |
| | on skin | | | | | | | | | | | |
| Dimethicone | Fragrance | 5 | 5 | 0 | 3 | 5 | 5 | 3 | 5 | 4 | 3.9 | 3.0 |
| | Texture | 2 | 4 | 2 | 3 | 2 | 4 | 2 | 3 | 2 | 2.7 | |
| | Initial | 2 | 4 | 1 | 3 | 3 | 2 | 2 | 4 | 2 | 2.6 | |
| | Skin Feel | | | | | | | | | | | |
| | Shine | 2 | 1 | 3 | 4 | 2 | 3 | 3 | 3 | 4 | 2.8 | |
| | Absorption | 2 | 4 | 4 | 4 | 3 | 3 | 4 | 2 | 3 | 3.2 | |
| | Skin feel on | 2 | 5 | 3 | 3 | 1 | 3 | 2 | 2 | 3 | 2.7 | |
| | application | | | | | | | | | | | |
| | After feel | 2 | 5 | 4 | 4 | 3 | 3 | 3 | 3 | 2 | 3.2 | |
| | Effect | 3 | 4 | 4 | 3 | 2 | 3 | 1 | 3 | 3 | 2.9 | |
| | on skin | | | | | | | | | | | |

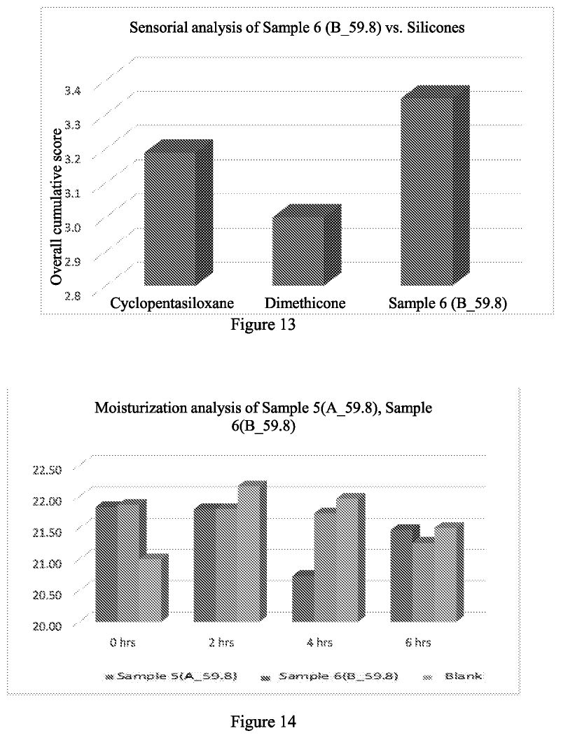

Sample 6 (B_59.8) had the overall cumulative score of 3.4 as against the silicones, with 3.2 for cyclopentasiloxanes and 3.0 for dimethicone (Table 18 & FIG. 13) However it can also be observed that the Sample 6 (B_59.8) have much lower score than Sample 2 (B_89.8) in fragrance and absorbance and the overall score is lesser for Sample 6 compared to Sample 2. This is because Sample 6 has higher coco caprylate caprate and lower MCT compared to Sample 2. This deviation in the weight % of the components did not provide in desired sensorial effect, though exhibited better sensorial effect than the silicones. Therefore, it is essential that a composition to exhibit an efficient sensorial property should have the components within said weight ranges as disclosed herein.

### Moisturization Analysis: Sample 6 (B_59.8)

Moisturization analysis of the Sample 6 (B_59.8) was evaluated and it was evident from FIG. 14 that the Sample 6 (B_59.8) showed decreasing moisturization abilities post 2 hours compared to blank. Similar to Sample 5 above, sample 6 also comprises lesser MCT % and higher coco caprylate caprate % in the composition compared to the disclosed weight ranges. This deviation in the weight % caused the non-synergistic moisturization ability though the composition exhibited a comparable sensorial effect. Thus these examples confirmed that the composition comprising the components falling within said weight ranges are critical in providing desired overall sensorial and moisturization effect on skin.

### Example 4

### Skin-Care and Hair-Care Composition

The present disclosure provides a topical composition for topical application on a keratinous tissue i.e for topical application on skin and hair. The skin-care composition comprises medium chain triglycerides with isopropyl myristate and vitamin E within said weight ranges as disclosed herein. Isopropyl myristate being a polar emollient is suitable for skin cosmetics and hence is preferred in a skin-care composition. Thus the skin-care composition comprises 89.8% MCT, 10% isopropyl myristate and 0.2% vitamin E.

A hair-care composition of the present disclosure comprises medium chain triglycerides with coco caprylate caprate and vitamin E within said weight ranges as disclosed herein. Coco caprylate caprate being a fast-spreading, low-greasiness emollient is suitable for hair care cosmetics and hence is preferred in a hair-care composition. Thus, the hair-care composition comprises 89.8% MCT, 10% coco caprylate caprate and 0.2% vitamin E. The potential glossiness and shining appearance that the hair-care composition could offer to hair was assessed using the gloss test. Refractive indices were determined in various angles and is tabulated in Table 19 below.

TABLE 19

| | Hair-care Composition | 20° | 60° | 85° |
|---|---|---|---|---|
| Sr. No. | Sample 2 (B_89.8) | value | value | value |
| 1 | Before Hair | 0.03 | 0.08 | 0.13 |
| | serum application | | | |
| 2 | After Hair | 0.04 | 0.2 | 0.21 |
| | serum application | | | |

As evident from the values in Table 19 the refractive indices of the hair-care sample remarkably increased post its application to a hair swatch, particularly at geometrical angles 60 and 85. Thus it is evident that sample 2 (B_89.8) can be effectively be used in hair care category to enhance hair shine, mitigate fly away and tangling of hair.

Overall, the topical composition of skin-care or hair-care composition comprising MCT in the weight percentage range of 70-95%, isopropyl myristate or coco caprylate caprate in the weight percentage range of 1-15%, and vitamin E in the weight percent range of 0.1-0.3% is crucial for achieving the desired composition of enhanced moisturizing and nourishing effect over skin and hair. Further, the specific working composition also exhibits enhanced moisturization effect than the silicones like cyclopentasiloxane, dimethicones. Any slight deviation from the disclosed weight ranges does not provide a composition with the desired sensorial and moisturization effect.

### ADVANTAGES OF THE PRESENT DISCLOSURE

The present disclosure focuses on the development of non-toxic, safe, biodegradable topical composition comprising medium chain triglycerides (MCT); isopropyl myristate or coco caprylate caprate; and Vitamin E. The composition of the present disclosure is based on the combination of the naturally-derived ingredients in the weight percentages as disclosed above that helps in the formation of a film on the skin that helps in enhancing the moisturizing and nourishing ability and providing an enhanced sensorial feel. The composition of the present disclosure is a silicon-free composition that circumvents the problems existing in the conventional cosmetic composition containing silicones. The present disclosure provides a simple composition with minimum of ingredients that are derived natural resources and hence is environmentally friendly product. Further the present disclosure provides a simple process for preparing the composition which makes the composition economically viable. The composition of the present disclosure is also found to be stable for at least 3 months and is found to be cytocompatible. The present disclosure also provides a cosmetic formulation with an additive for use in topical application over skin and hair. The composition of the present disclosure is useful in treating a keratinous tissue and providing enhanced moisturizing and nourishing effect to skin and hair.

1. A topical composition comprising:
a. 70 to 95% (w/w) of a triglyceride;
b. 1 to 15% (w/w) of an ester of C₈ to C₁₆ fatty acid; and
c. 0.1 to 0.3% (w/w) of vitamin E,
wherein the triglyceride is a medium chain triglyceride comprising C₆ to C₁₂ fatty acids.

2. The composition as claimed in claim 1, wherein the ester of C₈ to C₁₆ fatty acid is isopropyl myristate or caprylate caprate.

3. The composition as claimed in claim 1, wherein the triglyceride has an acid value of less than 0.1 and colour index of less than 50.

4. The composition as claimed in claim 1, wherein the composition is for skin-care or hair-care.

5. The composition as claimed in claim 1, wherein the composition is chemically stable for at least 3 months at 45° C., as measured at 70% RH.

6. The composition as claimed in claim 1, wherein the composition is stable and exhibits cytocompatibility.

7. The composition as claimed in claim 1, wherein the composition further comprises a component selected from antimicrobial, antibacterial, antifungal, antiprotozoal, and antiviral agents.

8. A process for preparing the composition as claimed in claim 1, the process comprising: contacting stoichiometric ratio of a triglyceride, an ester of C₈ to C₁₆ fatty acid, and vitamin E under stirring at a speed in a range of 50 to 1200 rpm at a temperature in a range of 20 to 60° C., wherein the triglyceride is a medium chain triglyceride comprising C₆ to C₁₂ fatty acids.

9. A cosmetic formulation comprising the composition as claimed in claim 1 with an additive.

10. The formulation as claimed in claim 9, wherein the additive is selected from a preservative, a pH adjusting agent, a chelating agent, or a co-solvent.

11. The formulation as claimed in claim 9 is topically applied on hair or skin.

12. A method of treating keratinous tissue of a subject, the method comprising topically applying the composition as claimed in claim 1 to the keratinous tissue.

13. A method of treating hair, the method comprising topically applying to hair in need thereof, the composition as claimed in claim 1 wherein topical application of the composition to the hair treats the hair.

14. A method of moisturizing skin, comprising applying an effective amount of the composition as claimed in claim 1 to the skin.

15. (canceled)

16. (canceled)

17. A skin-care topical composition comprising:
a. 70 to 95% (w/w) of a triglyceride;
b. 1 to 15% (w/w) of isopropyl myristate; and
c. 0.1 to 0.3% (w/w) of vitamin E,
wherein the triglyceride is a medium chain triglyceride comprising C₆ to C₁₂ fatty acids.

18. A hair-care topical composition comprising:
a. 70 to 95% (w/w) of a triglyceride;
b. 1 to 15% (w/w) of caprylate caprate; and
c. 0.1 to 0.3% (w/w) of vitamin E,
wherein the triglyceride is a medium chain triglyceride comprising C₆ to C₁₂ fatty acids.
